(19) United States
(12) Patent Application Publication
(10) Pub. No.: US 2022/0347241 A1
(43) Pub. Date: Nov. 3, 2022

(54) PREPARATION OF PHAGE COCKTAIL AS THERAPEUTIC AGENT FOR COW MASTITIS AND USE THEREOF

(71) Applicant: SHIHEZI UNIVERSITY, Xinjiang (CN)

(72) Inventors: Yan LIANG, Xinjiang (CN); Yonggang QU, Xinjiang (CN); Jie LI, Xinjiang (CN); Ruiyu YANG, Xinjiang (CN); Junshuai CHANG, Xinjiang (CN); Ge LIU, Xinjiang (CN); Liwei WANG, Xinjiang (CN); Qian ZHANG, Xinjiang (CN)

(73) Assignee: SHIHEZI UNIVERSITY, Xinjiang (CN)

(21) Appl. No.: 17/690,379

(22) Filed: Mar. 9, 2022

(30) Foreign Application Priority Data
Apr. 30, 2021 (CN) 202110481814.1

(51) Int. Cl. A61K 35/76; A61P 31/04; A61K 9/00

(52) U.S. Cl. CPC A61K 35/76; A61P 31/04; A61K 9/0019

(57) ABSTRACT

Disclosed is preparation of a cocktail as a therapeutic agent for cow mastitis and use thereof. A compatibility of phages against the main pathogenic bacteria of cow mastitis is utilized in combination with a specific preparation method to prepare and obtain a therapeutic cocktail preparation. The therapeutic phage cocktail preparation for cow mastitis provided in the disclosure is easy to prepare, short in course of treatment and quick in effect. It has better therapeutic effect on both clinical cow mastitis and recessive mastitis than that of antibiotics and antimicrobial peptide and is expected to solve the problem of bacterial resistance in the treatment of cow mastitis, thereby reducing or getting rid of the troubles of diseases such as mastitis in cows, which is of potential development value in cow farming.

### CROSS REFERENCE TO RELATED APPLICATION

This patent application claims the benefit and priority of Chinese Patent Application No. 202110481814.1 filed on Apr. 30, 2021, the disclosure of which is incorporated by reference herein in its entirety as part of the present application.

### TECHNICAL FIELD

The disclosure belongs to the technical field of animal disease prevention and control, and relates to the technical field of preparation of a microbial preparation, in particular to a therapeutic phage cocktail preparation for cow mastitis, methods of making the same and use thereof.

### BACKGROUND ART

Cow mastitis is a common and frequently-occurring disease in cows, which is most harmful to cow breeding. It is one of the main diseases of cows with the most input of medicines and the most difficult prevention and treatment, causing huge economic losses every year. At present, the control of cow mastitis is still dominated by treatment with traditional antibiotics and antibacterial drugs. The long-term use of antibiotics and antibacterial drugs has led to ever stronger bacterial resistance. The analysis of the drug resistance and sensitivity of cow mastitis pathogens in China has shown that cow mastitis pathogens exhibits a high drug resistance to most traditional drugs such as drugs belonging to penicillin, sulfonamides, aminoglycosides, tetracycline and macrolides, and are sensitive to quinolone and cephalosporin. Meanwhile, the drug residues caused by traditional methods are even more serious. Due to the emergence of bacterial resistance, clinical veterinarians often increase the dose to achieve a better therapeutic effect. Large doses of drugs directly injected into the nipple inevitably cause drug residues in milk products, which seriously affects the quality of milk products.

In order to better solve the clinical problems incurred by this phenomenon, it has become a research hotspot to seek new treatment methods for cow mastitis. Phages are small in size, simple in structure, have strong vitality and are present widely in the environment. Wherever bacteria grow and reproduce, phages may exist. Phages are highly specific and only target the corresponding pathogenic bacteria. It kills bacteria in a way host-specific, without disordering the normal flora and causing serious infection. Phages have a strong ability to reproduce, and are not prone to develop bacterial resistance. At the same time, as a microecological preparation, the phage preparation renders no drug residue. Phages, as an antibacterial microecological preparation, are once again in the spotlight. At present, phage therapy is still immature in clinical use. Certain phages are effective in a specific in vitro test environment, but it does not mean that they are effective in vivo. Studies have also shown that some phages must enter the mammary glands of cows and come into contact with raw milk in order to play a better role, the in vivo therapeutic effect of which is not ideal. In addition, immune globulins of diseased cows have an inhibitory effect on phages. Simultaneously, Staphylococcus aureus forms a thick biofilm in abscessed cow udders to inhibit the entry of phages, which may also lead to poor therapeutic effects. Dosage and timing of phage therapy are also important factors that affect the efficacy of the therapy. Phages only begin to proliferate when the bacteria reach a certain density, and phages inoculated too early or at an inappropriate dose may be eliminated by the body before they begin to proliferate. Therefore, combining specific formulations with determination of the optimal inoculation time and dose becomes a major difficulty in phage therapy. Hence, before large-scale application of phage to the treatment of cow mastitis, further research is needed to clarify its efficacy, safety and administration methods.

### SUMMARY

In view of the problems in the prior art that the mixed phages in the existing phage preparations for treatment of cow mastitis are poorly selected, the lysis spectrum is narrow, and the therapeutic effect for clinical mastitis and recessive mastitis is undesirable, a variety of lytic phages obtained by screening are used in the present disclosure to prepare a therapeutic cocktail preparation, aiming at effectively treating cow mastitis and improving the added value of products. An objective of the present disclosure is to provide a therapeutic phage cocktail preparation for cow mastitis, which is easy to prepare, has a short course of treatment and quick effects. The preparation has good therapeutic effect for both clinical mastitis and recessive mastitis, and the therapeutic effect on diseased cows is better than that of the related antimicrobial peptide treatment and antibiotic treatment. It is expected to solve the problem of bacterial resistance in the treatment of cow mastitis, thereby reducing or getting rid of the troubles of cow mastitis and the like.

The present disclosure is implemented through the following technical solutions:

The present disclosure specifically provides a therapeutic phage cocktail preparation for cow mastitis, in which the therapeutic phage cocktail preparation for cow mastitis includes a cow mastitis-derived Escherichia coli phage selected from the group consisting of Ecp1, Ecp3, Ecp5, Ecp16, Ecp17, and vB_EcoM_XJ2, a cow mastitis-derived Streptococcus phage selected from the group consisting of vB_StrM_L1 and vB_SagS_FSN1, and a cow mastitis-derived Staphylococcus aureus phage selected from the group consisting of P42 and vB_SauS_IMEP5.

In some embodiments, the cow mastitis-derived E. coli phage, the cow mastitis-derived Streptococcus phage, and the cow mastitis-derived S. aureus phage in the therapeutic phage cocktail preparation for cow mastitis are mixed in a volume ratio of 1:1:1.

In some embodiments, the cow mastitis-derived E. coli phages Ecp1, Ecp3, Ecp5, Ecp16, Ecp17, and vB_EcoM_XJ2 in the therapeutic phage cocktail preparation for cow mastitis are mixed in a volume ratio of 1:1:1:1:1:1.

In some embodiments, the cow mastitis-derived Streptococcus phages vB_StrM_L1 and vB_SagS_FSN1 in the therapeutic phage cocktail preparation for cow mastitis are mixed in a volume ratio of 1:1.

In some embodiments, the cow mastitis-derived S. aureus phages P42 and vB_Sau S_IMEP5 in the therapeutic phage cocktail preparation for cow mastitis are mixed in a volume ratio of 1:1.

Further, the present disclosure specifically provides a method for preparing the therapeutic phage cocktail preparation for cow mastitis, and the specific steps are as follows:

(1) selecting a frozen cow mastitis-derived E. coli phage selected from the group consisting of Ecp1, Ecp3, Ecp5, Ecp16, Ecp17, and vB_EcoM_XJ2, a frozen cow mastitis-derived Streptococcus phage selected from the group consisting of vB_StrM_L1 and vB_SagS_FSN1, and a frozen cow mastitis-derived S. aureus phage selected from the group consisting of P42 and vB_SauS_IMEP5 for recovery;

(2) taking 100 μL of a phage stock solution of each strain recovered in step (1), inoculating each solution into 5 mL of a host bacterial liquid in a logarithmic growth phase respectively, shaking and culturing at 37° C. at 180 r/min until turning clear, and centrifuging the solutions at 10,000 r/min for 10 min; taking each supernatant and obtaining phase dilutions with phosphate buffered solution at a concentration of 1×10⁹ pfu/mL; and

(3) mixing the phage dilutions of each strain prepared in step (2) in proportion to prepare a therapeutic phage cocktail preparation for cow mastitis.

The disclosure provides a use of the therapeutic phage cocktail preparation for cow mastitis prepared by the above process in the treatment of cow mastitis.

In the use of the therapeutic phage cocktail preparation for cow mastitis in the treatment of cow mastitis, a method of administration includes directly injecting the phage cocktail into a gland cistern through a teat canal.

In the use of the therapeutic phage cocktail preparation for cow mastitis in the treatment of cow mastitis, a dosage thereof is 30 mL each for 2-3 times a day.

In the present disclosure, the 10 strains of phages, i.e., the cow mastitis-derived E. coli phages ECp1, Ecp3, Ecp5, Ecp16, Ecp17, vB_EcoM_XJ2, the cow mastitis-derived Streptococcus phages vB_StrM_L1, vB_SagS_FSN1, and the cow mastitis-derived S. aureus phages P42, vB_SauS_IMEP5, are all well-known phages, which can be obtained by those skilled in the art through publicly available channels, and the culture conditions and media of the above 10 kinds of phages are the same as those commonly reported in the art.

According to the embodiments of the present disclosure, the following beneficial effects may be achieved:

(1) In the present disclosure, a compatibility of phages against the main pathogenic bacteria of cow mastitis is utilized in combination with a specific preparation method to prepare and obtain a therapeutic cocktail preparation, aiming to improve the added value of the product in addition to prevention and treatment. The therapeutic phage cocktail preparation for cow mastitis provided in the disclosure is easy to prepare, short in course of treatment and quick in effect and has a good therapeutic effect on both clinical cow mastitis and recessive mastitis. It is expected to solve the problem of bacterial resistance in the treatment of cow mastitis, thereby reducing or getting rid of the troubles of diseases such as mastitis in cows.

(2) When the cocktail preparation prepared according to the embodiments of the present disclosure is applied to the treatment of clinical mastitis in cows, Somatic Cell Counts (SCC) in the milk samples of the diseased cows after treatment are significantly reduced, the cure rate of the diseased cows is 8.34% higher than that of the antibiotic treatment group, and the cure rate of the mammary regions is 3.57% higher than that of the antibiotic treatment group; when the cocktail preparation prepared according to the embodiments of the present disclosure is applied to the treatment of cow recessive mastitis, the cure rate of the diseased cow is 8.33% higher than that of the antimicrobial peptide treatment group, the same in effect to that of the antibiotic treatment group, and the cure rate of the mammary regions is 7.5% higher than that of the antimicrobial peptide treatment group and 1.79% higher than that of the antibiotic treatment group. The therapeutic phage cocktail preparation for cow mastitis prepared in the disclosure is of potential development value in the field of cow breeding.

### BRIEF DESCRIPTION OF THE DRAWINGS

FIG. 1 shows the clinical therapeutic effect of the therapeutic phage cocktail preparation for cow mastitis.

FIG. 2 shows the effect of phage cocktail as a therapeutic agent for cow recessive mastitis.

### DETAILED DESCRIPTION OF THE EMBODIMENTS

Hereinafter, the present disclosure will be described with reference to examples, but the present disclosure is not limited to the following examples.

The reagents used in the present disclosure include: PEG8000 purchased from Tianjin Guangfu Fine Chemical Research Institute; SM buffer: NaCl 5.8 g, MgSO₄.7H₂O 2.0 g, 1 mol/L Tris-HCl (pH 7.5) 50 mL, 2% gelatin 5 mL, a balance of distilled water to 1 L, sterilized at 1×10Pa for 20 min, and stored at 4° C.; PBS buffer: NaCl 8.0 g, KCl 0.2 g, Na₂HPO₄ 1.42 g, KH₂PO₄ 0.27 g, a balance of distilled water to 1 L, pH adjusted to 7.4, sterilized at 1×10Pa for 20 min, and stored at 4° C.

The instruments and equipment used in the present disclosure are: DNP-9082 electro-thermostatic incubator from Shanghai Jinghong Experimental Equipment Co., Ltd.); MIKRO 120 high-speed desktop centrifuge from German HETTICH manufacturing company); Multifuge X1R desktop refrigerated centrifuge from Thermo Fisher Scientific (China) Co., Ltd.; HVE-50 autoclave from Japan HIRAYAMA company; ZHWY-2102 double-layer thermostatic shaker, Shanghai Zhicheng company; YHC-260 medicine storage cabinet from Shanghai Touching Technology Co., Ltd., and so on.

The reagents and materials are commercially available, and the equipment and instruments used in the process are common equipment in the art.

All materials, reagents and instruments used in the present disclosure are well known in the art, but do not limit the implementation of the present disclosure, and some other reagents and equipment well known in the art are suitable to the following embodiments of the present disclosure.

The following examples further illustrate the embodiments of the present disclosure but should not be construed as limiting the present disclosure. Modifications or substitutions made to the methods, steps or conditions of the present disclosure without departing from the spirit and essence of the present disclosure all fall within the scope of the present disclosure.

### Example 1: Therapeutic Phage Cocktail Preparation for Cow Mastitis

The present disclosure specifically provided a therapeutic phage cocktail preparation for cow mastitis, and the therapeutic phage cocktail preparation for cow mastitis included cow mastitis-derived E. coli phages Ecp1, Ecp3, Ecp5, Ecp16, Ecp17, and vB_EcoM_XJ2, cow mastitis-derived Streptococcus phages vB_StrM_L1 and vB_SagS_FSN1, and cow mastitis-derived S. aureus phages P42 and vB_SauS_IMEP5.

In the therapeutic phage cocktail preparation for cow mastitis, the cow mastitis-derived E. coli phages, the cow mastitis-derived Streptococcus phages, and the cow mastitis-derived S. aureus phages were mixed in a volume ratio of 1:1:1.

In the therapeutic phage cocktail preparation for cow mastitis, cow mastitis-derived E. coli phages Ecp1, Ecp3, Ecp5, Ecp16, Ecp17, and vB_EcoM_XJ2 were mixed in a volume ratio of 1:1:1:1:1:1.

In the therapeutic phage cocktail preparation for cow mastitis, the cow mastitis-derived Streptococcus phages vB_StrM_L1 and vB_SagS_FSN1 were mixed in a volume ratio of 1:1.

In the therapeutic phage cocktail preparation for cow mastitis, the cow mastitis-derived S. aureus phage P42 and vB_Sau S_IMEP5 were mixed in a volume ratio of 1:1.

### Example 2: Method for Preparing Therapeutic Phage Cocktail Preparation for Cow Mastitis

The disclosure specifically provided a method for preparing the therapeutic phage cocktail preparation for cow mastitis, and the specific steps were as follows:

(1) frozen cow mastitis-derived E. coli phages Ecp1, Ecp3, Ecp5, Ecp16, Ecp17, and vB_EcoM_XJ2, cow mastitis-derived Streptococcus phages vB_StrM_L1 and vB_SagS_FSN1, and cow mastitis-derived S. aureus phages P42 and vB_Sau S_IMEP5 were selected for recovery;

(2) 100 μL of the phage stock solution of each strain recovered in step (1) was inoculated into 5 mL of a host bacterial liquid in a logarithmic growth phase respectively, then shaken and cultured at 37° C. at 180 r/min until each solution became clear, and centrifuged at 10,000 r/min for 10 min; a resulting supernatant was taken and diluted with phosphate buffered solution to a concentration of 1×10⁹ pfu/mL to obtain phase dilutions;

(3) the phage dilutions of each strain prepared in step (2) were mixed in the proportion according to Example 1 to prepare a therapeutic phage cocktail preparation for cow mastitis.

### Example3: Therapeutic Phage Cocktail Preparation for Cow Mastitis

The disclosure specifically provided a method for preparing the therapeutic phage cocktail preparation for cow mastitis, and the specific steps were as follows:

(1) frozen cow mastitis-derived E. coli phages Ecp1, Ecp3, Ecp5, Ecp16, Ecp17, and vB_EcoM_XJ2, and cow mastitis-derived Streptococcus phages vB_StrM_L1 and vB_SagS_FSN1 were selected for recovery;

(2) 100 μL of a phage stock solution of each strain obtained by purification and screening in step (1) was inoculated into 5 mL of a host bacterial liquid in logarithmic growth phase respectively, shaken and cultured at 37° C. at 180 r/min until each solution became clear, and centrifuged at 10000 r/min for 10 min; a resulting supernatant was taken and diluted to a concentration of 1×10 ⁹ pfu/mL to obtain phase dilutions;

(3) the phage dilutions of each strain prepared in step (2) were mixed in the proportion according to Example 1 to prepare a therapeutic phage cocktail preparation for cow mastitis.

### Example 4: Therapeutic Phage Cocktail Preparation for Cow Mastitis

The disclosure specifically provided a method for preparing a therapeutic phage cocktail preparation for cow mastitis, and the specific steps were as follows:

(1) frozen cow mastitis-derived E. coli phages Ecp1, Ecp3, Ecp5, Ecp16, Ecp17, and vB_EcoM_XJ2, and cow mastitis-derived S. aureus phages P42 and vB_SauS_IMEP5 for recovery;

(2) 100 μL of the phage stock solution of each strain obtained by purification and screening in step (1) was inoculated into 5 mL of a host bacterial liquid in logarithmic growth phase respectively, shaken and cultured at 37° C. at 180 r/min until each solution became clear, and centrifuged at 10000 r/min for 10 min; a resulting supernatant was taken and diluted to a concentration of 1×10⁹ pfu/mL to obtain phase dilutions;

(3) the phage dilutions of each strain prepared in step (2) were mixed in the proportion according to Example 1 to prepare a therapeutic phage cocktail preparation for cow mastitis.

### Example 5: Therapeutic Phage Cocktail Preparation for Cow Mastitis

The disclosure specifically provided a method for preparing a therapeutic phage cocktail preparation for cow mastitis, and the specific steps were as follows:

(1) frozen bovine mastitis-derived Streptococcus phages vB_StrM_L1, and vB_SagS_FSN1, and cow mastitis-derived S. aureus phages P42 and vB_SauS_IMEP5 were selected for recovery;

(2) 100 μL of the phage stock solution of each strain obtained by purification and screening in step (1) was inoculated into 5 mL of a host bacterial liquid in logarithmic growth phase respectively, shaken and cultured at 37° C. at 180 r/min until each solution became clear, and centrifuged at 10000 r/min for 10 min; a resulting supernatant was taken and diluted to a concentration of 1×10⁹ pfu/mL to obtain phase dilutions;

(3) the phage dilutions of each strain prepared in step (2) were mixed in the proportion according to Example 1 to prepare a therapeutic phage cocktail preparation for cow mastitis.

### Example 6: Determination of Effect of Different Mixing Ratios of Phages

Phage cocktails as therapeutic agent for cow mastitis were prepared according to the Examples 2 to 5. After each group of phage preparations were diluted with PBS, they were added to a mixed suspension of main pathogenic bacteria of cow mastitis (E. coli, S. aureus, and Streptococcus). The growth of plaque was observed by double-layer agar method, and the experimental design of each group are shown in Table 1.

TABLE 1
Experimental Design

| Group | Culture |
|---|---|
| Control | Suspension of main pathogens of cow mastitis |
| Treatment | therapeutic phage cocktail preparation for cow mastitis |
| group 1 | prepared in Example 3 |
| Treatment | therapeutic phage cocktail preparation for cow mastitis |
| group 2 | prepared in Example 4 |
| Treatment | therapeutic phage cocktail preparation for cow mastitis |
| group 3 | prepared in Example 5 |
| Treatment | therapeutic phage cocktail preparation for cow mastitis |
| group 4 | prepared in Example 2 |

According to the experimental design in the above table 1, the growth of plaques in each group was observed. No plaque appeared in the control group. The number of plaques in treatment group 4 was the largest. The numbers of plaques in treatment groups 1, 2 and 3 were lower than that of treatment group 4, which showed little difference among the groups. Therefore, it could be seen that in the preparation of the therapeutic phage cocktail preparation for cow mastitis, the combination of cow mastitis-derived E. coli phages, cow mastitis-derived Streptococcus phages and cow mastitis-derived S. aureus phages had the best lysis effect on the main pathogenic bacteria of cow mastitis.

### Example 7: Determination of Therapeutic Effect of Therapeutic Phage

### Cocktail Preparation for Cow Mastitis

Based on the screening results of Example 6, the therapeutic effect of the prepared therapeutic phage cocktail preparation for cow mastitis was determined.

### (1) Experimental Grouping

1. The therapeutic phage cocktail preparation for cow mastitis prepared according to Example 2 with the best in vitro lysis effect was used to perfuse the udders of diseased cows with mastitis as a phage treatment group. A concentration of 1×10⁹-1×10⁸ pfu/mL phage was directly injected into the gland cistern through the teat canal, with a dosage of 30 mL each for 2-3 times a day for 1-2 weeks.

2. Commercially available antimicrobial peptide products were used to perfuse the udders of cows with mastitis as the antimicrobial peptide treatment group, which was directly injected into the gland cistern through the teat canal. The dosage was 30 mL each for 2-3 times a day for 1-2 weeks.

3. Routine treatment with antibiotics (penicillin sodium and streptomycin sulfate) was set as the antibiotic treatment group for control.

### (2) Determination of Therapeutic Effect

The typical clinical mastitis and recessive mastitis cases were selected, and the lytic phage or lyase gene expression product was injected into the udder by an udder perfusion method, twice a day, for 1-2 weeks; at the same time, the clinical routine drug treatment group was selected as control. The California Mastitis Test (CMT) was used to measure the incidence of recessive mastitis and the level of SCC in milk samples of the tested cows. Changes in the disease incidence and in the detected negative number of head and mammary region with mastitis across time periods were the main indexes for judgment. The measurement results are shown in Table 2.

TABLE 2
Determination of the effect of different regimens on cow mastitis.

| | Clinical mastitis | | Recessive mastitis | |
|---|---|---|---|---|
| | treatment | | treatment | |
| | Cure rate | Cure rate | Cure rate | Cure rate |
| | of diseased | of mammary | of diseased | of mammary |
| Treatment | cow | region | cow | region |
| Phage | 66.67% | 75.00% | 83.33% | 87.50% |
| treatment | | | | |
| Antimicrobial | Not | Not | 75.00% | 80.00% |
| peptide | conducted | conducted | | |
| treatment | | | | |
| Antibiotic | 58.33% | 71.43% | 83.33% | 85.71% |
| treatment | | | | |

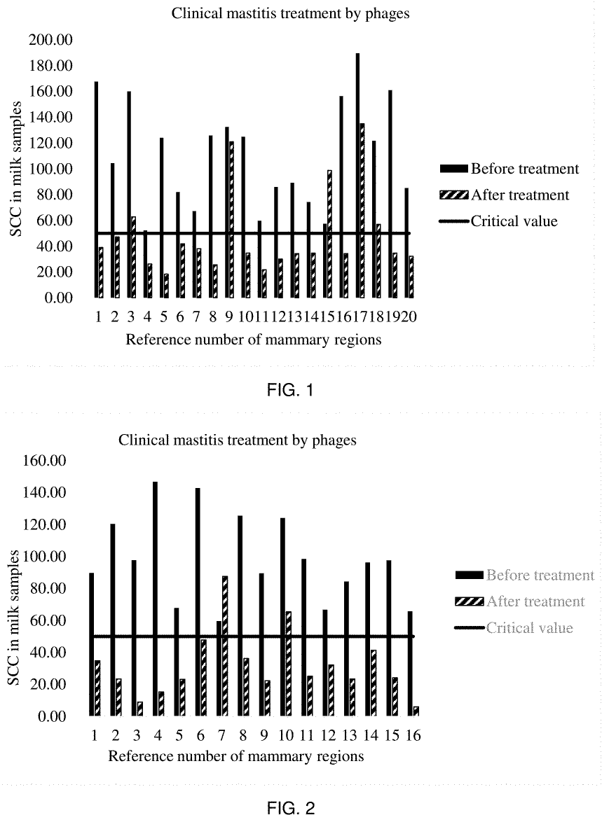

The cocktail preparation prepared according to the embodiments of the present disclosure was applied to the treatment of clinical mastitis in cows. After treatment, the SCC in the milk sample of the diseased cow was significantly reduced. The specific determination results are shown in FIG. 1 and FIG. 2. As shown by the measurement results in the above Table 2, the therapeutic effect of the therapeutic phage cocktail preparation for cow mastitis provided in the present disclosure was better than that of the antimicrobial peptide treatment group and the antibiotic treatment group for control. The cocktail preparation prepared from 6 strains of cow mastitis-derived E. coli phage, 2 strains of cow mastitis-derived S. aureus phage, and 2 strains of cow mastitis-derived Streptococcus phage, and the teat perfusion method were used to treat cow mastitis caused by E. coli, S. aureus, and Streptococcus. The cure rate of clinical mastitis of diseased cows by phages was 66.67% (8/12), and the cure rate of clinical mastitis of mammary regions was 75.00% (15/20); the cure rate of recessive mastitis of diseased cows was 83.33% (10/12), and the cure rate in mammary regions was 87.50% (14/16); of the 7 mammary regions that were not cured, 5 were mammary regions from cows with clinical mastitis, and 2 were mammary regions from cows with recessive mastitis. The control group was treated with antibiotics (penicillin sodium and streptomycin sulfate). For clinical cow mastitis, the cure rate was 58.33% (7/12) in the diseased cows, and 71.43% (15/21) in the mammary regions. For recessive cow mastitis, the cure rate was 83.33% (10/12) in diseased cows and 85.71% (18/21) in the mammary regions. Statistical analysis showed that there was a significant difference in the cure rate of the two treatments for cows with clinical mastitis (P=0.033, P<0.05); the cure rate for the recessive mastitis was not significantly different (P=0.087, P>0.05).

The present disclosure will be well implemented according to the embodiments above. The above-mentioned embodiments are only to describe the preferred embodiments of the present disclosure, and do not limit the scope of the present disclosure. Various changes and improvements to the technical solutions of the present disclosure made by those skilled shall fall within the protection scope determined by the present disclosure.

What is claimed is:

1. A therapeutic phage cocktail preparation for cow mastitis, wherein the therapeutic phage cocktail preparation for cow mastitis comprises a cow mastitis-derived Escherichia coli phage selected from the group consisting of Ecp1, Ecp3, Ecp5, Ecp16, Ecp17, and vB_EcoM_XJ2, a cow mastitis-derived Streptococcus phage selected from the group consisting of vB_StrM_L1 and vB_SagS_FSN1, and a cow mastitis-derived Staphylococcus aureus phage selected from the group consisting of P42 and vB_SauS_IMEP5.

2. The therapeutic phage cocktail preparation for cow mastitis of claim 1, wherein the cow mastitis-derived E. coli phage, the cow mastitis-derived Streptococcus phage, and the cow mastitis-derived S. aureus phage in the therapeutic phage cocktail preparation for cow mastitis are mixed in a volume ratio of 1:1:1.

3. The therapeutic phage cocktail preparation for cow mastitis of claim 1, wherein the cow mastitis-derived E. coli phages Ecp1, Ecp3, Ecp5, Ecp16, Ecp17, and vB_EcoM_XJ2 in the therapeutic phage cocktail preparation for cow mastitis are mixed in a volume ratio of 1:1:1:1:1:1.

4. The therapeutic phage cocktail preparation for cow mastitis of claim 1, wherein the cow mastitis-derived Streptococcus phages vB_StrM_L1 and vB_SagS_FSN1 in the therapeutic phage cocktail preparation for cow mastitis are mixed in a volume ratio of 1:1.

5. The therapeutic phage cocktail preparation for cow mastitis of claim 1, wherein the cow mastitis-derived S. aureus phages P42 and vB_Sau S_IMEP5 in the therapeutic phage cocktail preparation for cow mastitis are mixed in a volume ratio of 1:1.

6. A method for preparing the therapeutic phage cocktail preparation for cow mastitis of claim 1, wherein the method comprises following steps:
(1) selecting a frozen cow mastitis-derived E. coli phage selected from the group consisting of Ecp1, Ecp3, Ecp5, Ecp16, Ecp17, and vB_EcoM_XJ2, a frozen cow mastitis-derived Streptococcus phage selected from the group consisting of vB_StrM_L1 and vB_SagS_FSN1, and a frozen cow mastitis-derived S. aureus phage selected from the group consisting of P42 and vB_SauS_IMEP5 for recovery;
(2) taking 100 μL of a phage stock solution of each strain recovered in step (1), inoculating each solution into 5 mL of a host bacterial liquid in a logarithmic growth phase respectively, shaking and culturing at 37° C. at 180 r/min until turning clear, and centrifuging the solutions at 10,000 r/min for 10 min; taking each supernatant and obtaining phase dilutions with phosphate buffered solution at a concentration of 1×10⁹ pfu/mL; and
(3) mixing the phage dilutions of each strain prepared in step (2) in proportion to prepare a therapeutic phage cocktail preparation for cow mastitis.

7. A method for treating cow mastitis, comprising administering the therapeutic phage cocktail preparation for cow mastitis of claim 1 to a cow in need thereof.

8. The method of claim 7, wherein the administering comprises directly injecting the phage cocktail into a gland cistern through a teat canal for perfusion.

9. The method of claim 7, wherein a dosage is 30 mL each for 2-3 times a day.

10. The method of claim 7, wherein the cow mastitis-derived E. coli phage, the cow mastitis-derived Streptococcus phage, and the cow mastitis-derived S. aureus phage in the therapeutic phage cocktail preparation for cow mastitis are mixed in a volume ratio of 1:1:1.

11. The method of claim 7, wherein the cow mastitis-derived E. coli phages Ecp1, Ecp3, Ecp5, Ecp16, Ecp17, and vB_EcoM_XJ2 in the therapeutic phage cocktail preparation for cow mastitis are mixed in a volume ratio of 1:1:1:1:1:1.

12. The method of claim 7, wherein the cow mastitis-derived Streptococcus phages vB_StrM_L1 and vB_SagS_FSN1 in the therapeutic phage cocktail preparation for cow mastitis are mixed in a volume ratio of 1:1.

13. The method of claim 7, wherein the cow mastitis-derived S. aureus phages P42 and vB_Sau S_IMEP5 in the therapeutic phage cocktail preparation for cow mastitis are mixed in a volume ratio of 1:1.
